(19) United States
(12) Patent Application Publication
(10) Pub. No.: US 2019/0142336 A1
(43) Pub. Date: May 16, 2019

(54) SYSTEMS AND METHODS FOR DETERMINING RESPONSE TO ANESTHETIC AND SEDATIVE DRUGS USING MARKERS OF BRAIN FUNCTION

(71) Applicant: THE GENERAL HOSPITAL CORPORATION, Boston, MA (US)

(72) Inventors: Patrick L. Purdon, Somerville, MA (US); Emery N. Brown, Boston, MA (US); Yu Shao, Boston, MA (US)

(21) Appl. No.: 16/302,017

(22) PCT Filed: May 19, 2017
(86) PCT No.: PCT/US17/33618

§ 371 Date: Nov. 15, 2018

Related U.S. Application Data
(60) Provisional application No. 62/339,042, filed on May 19, 2016.

(51) Int. Cl. A61B 5/00; A61B 5/04; A61B 5/0476; G06K 9/00

(52) U.S. Cl. CPC A61B 5/4821; A61B 5/04012; A61B 5/4839; G06K 9/0053; A61B 5/0476

(57) ABSTRACT

Systems and methods for determining a response of a patient to the administration of at least one drug having anesthetic properties are provided. In one aspect, a method includes receiving physiological data acquired from a patient, and analyzing the physiological data to determine at least one indicator of brain function. The method also includes determining, based on the at least one indicator of brain function, a response of the patient to the administration of at least one drug having anesthetic properties, and generating a report indicative of the response.

### CROSS-REFERENCE TO RELATED APPLICATIONS

This application is based on, claims priority to, and incorporates herein by reference in their entirety U.S. Ser. No. 62/339,042 filed May 19, 2016, and entitled “SYSTEMS AND METHODS FOR PREDICTING AND ESTIMATING THE DOSE RESPONSE OF ANESTHETIC AND SEDATIVE DRUGS USING MARKERS OF BRAIN FUNCTION.”

### GOVERNMENT RIGHTS

This invention was made with government support under DP2-OD006454, TR01-GM104948, and T32GM007592 awarded by National Institutes of Health. The government has certain rights in the invention.

### BACKGROUND

The present disclosure generally relates to anesthesia and sedation. More particularly, the present disclosure is directed to systems and methods for determining patient response to general anesthetic and sedative drugs using markers of brain function.

In the United States, nearly 60,000 patients receive general anesthesia per day to safely undergo surgical procedures. A large fraction of these patients are elderly at 60 years of age or older. Unlike treatment of younger patients, anesthetic management of older patients necessitates additional care and carries higher risks. For example, the doses of anesthetics required to achieve the same level of general anesthesia in elderly patients are 10 to 50 percent less compared to those for younger patients. Also, changes in heart rate and blood pressure are more likely to occur in older patients following induction of general anesthesia by bolus administration of a hypnotic. This requires clinical measures to be routinely taken to prevent the consequences of these changes.

Post-operative conditions in the elderly following general anesthesia and sedation are also a growing concern. For instance, delirium is an acute form of dysfunction whose symptoms include disorientation, impairment of attention and memory, while post-operative cognitive dysfunction (“POCD”) is a persistent cognitive disorder that lasts from a few hours to several days or months. Specifically, POCD can range from difficulty with fact-finding and memory impairment to dementia and Alzheimer's-like symptoms. In addition, the prevalence of more subtle forms of POCD, which may go undetected without formal neuropsychological testing, may be greater than currently appreciated. Although it is presently unclear as to what degree anesthesia and sedation influence such conditions, as population ages, the fraction of the elderly patients who will require therapeutic and diagnostic procedures will continue to increase.

Changes in the brain's gross anatomy over the course of normal aging include prominent loss of volume and thickness in the prefrontal cortex, particularly in the dorsal medial and dorsal lateral prefrontal cortices, as well as the lateral parietal and lateral temporal cortices. Such loss of volume and thickness in the prefrontal cortical regions, which play prominent roles in attention and executive function, is consistent with the findings from numerous psychological experiments showing age-related decreases in performance on tests of attention and executive function. Prominent changes that have been reported in the viscerosensory region of the caudal insular cortex also appear to undergo relatively prominent thinning with normal aging. Other investigations into loss of thickness or volume in other brain regions, such as the primary sensory and motor cortices, paralimbic and limbic areas, hippocampus and entorhinal cortex, and the cingulate and insula, have provided mixed results.

Post-operative delirium (POD) occurs frequently in elderly patients undergoing anesthesia. Recent studies have shown that burst suppression, assessed using electroencephalogram (EEG) measurements, is correlated with POD and impairment of functional independence. Burst suppression occurs at very deep levels of general anesthesia, or after prolonged sedation in the intensive care unit, and is associated with coma. As such, burst suppression in most cases is regarded as a state of anesthetic overdose beyond what is required for general anesthesia and sedation. In addition, aging is associated with significant neurobiological and neurophysiological changes that could render some elderly patients more vulnerable to burst suppression, and subsequently POD and cognitive dysfunction.

As appreciated from the above, there continues to be a clear need for new approaches in anesthesiology that take into account brain function in order to reduce or eliminate undesired effects, such as poor cognitive outcomes associated with inappropriate dosing.

### SUMMARY

The present disclosure provides systems and methods directed to anesthesia or sedation. In contrast to previous methods that rely on calendar age for example to estimate anesthesia dosing, the present disclosure introduces a novel approach based on brain function. In particular, using various signatures or markers of brain function, anesthetic dose response and drug requirements can be estimated and predicted. In addition, indicators related to poor cognitive outcomes, such as a likelihood of burst suppression, can also be determined using systems and methods described herein.

In accordance with one aspect of the disclosure, a method for determining a response of a patient to the administration of at least one drug having anesthetic properties is provided. The method includes receiving physiological data acquired from a patient, and analyzing the physiological data to determine at least one indicator of brain function. The method also includes determining, based on the at least one indicator of brain function, a response of the patient to the administration of at least one drug having anesthetic properties, and generating a report indicative of the response.

In accordance with another aspect of the disclosure, a system for determining a response of a patient to the administration of at least one drug having anesthetic properties is provided. The system includes a plurality of sensors configured to acquire physiological data from the patient, and a processor programmed to execute instructions stored in a non-transitory computer-readable medium to receive the physiological data, and analyze the physiological data to determine at least one indicator of brain function. The processor is also programmed to determine, based on the at least one indicator of brain function, a response of the patient to the administration of at least one drug having anesthetic properties, and generate a report indicative of the response. The system further includes an output for providing the report.

The foregoing and other advantages of the invention will appear from the following description.

### BRIEF DESCRIPTION OF THE DRAWINGS

The present invention will hereafter be described with reference to the accompanying drawings, wherein like reference numerals denote like elements. The patent or application file contains at least one drawing executed in color. Copies of this patent or patent application publication with color drawing(s) will be provided by the Office upon request and payment of the necessary fee.

FIG. 1B is schematic block diagram of an example physiological monitoring system, in accordance with aspects of the present disclosure.

FIG. 1B is schematic block diagram of another example physiological monitoring systems, in accordance with aspects of the present disclosure.

FIG. 2 is an illustration of an example monitoring and control system, in accordance with aspects of the present disclosure.

FIG. 3 is a flowchart setting forth steps of a process, in accordance with aspects of the present disclosure.

FIG. 4 is another flowchart setting forth steps of a process, in accordance with aspects of the present disclosure.

FIG. 5 is an illustration of how age information can be used to adjust the scales for displays of EEG information used to monitor anesthesia, in accordance with the present disclosure.

FIG. 6 is yet another flowchart setting forth steps of a process, in accordance with aspects of the present disclosure.

FIG. 7 is an illustration of EEG spectrograms during propofol-induced general anesthesia across a range of patient ages, in accordance with the present disclosure.

FIG. 8 is an illustration of how EEG spectral features may be associated with different dose responses and likelihoods of experiencing burst suppression, in accordance with the present disclosure.

FIG. 9 is a table that illustrates the use of logistic regression models to predict burst suppression using different covariates or measurements, demonstrating that at least EEG alpha power can be used to predict burst suppression and anesthetic drug sensitivity

FIG. 10 is a schematic that illustrates how the likelihood of burst suppression or drug sensitivity can be provided as an odds ratio.

FIG. 11 is an illustration of EEG spectrograms demonstrating how alpha power is associated with different dose responses and likelihoods of experiencing burst suppression, in accordance with the present disclosure.

FIG. 12 is a graph showing the relationship between the probability of burst suppression and EEG alpha power for the anesthetic drug propofol.

FIG. 13 is a graph showing the relationship between the probability of burst suppression and EEG alpha power for the anesthetic drug sevoflurane.

### DETAILED DESCRIPTION

Currently, anesthesia and sedation are applied in the clinic using various formulaic approaches that include taking into consideration patient age, weight, and other external factors. However, these approaches ignore a patient's individual brain function, which affects response to anesthetics and may not correspond to such broad characteristics. In fact, often observed side effects, such as post-operative cognitive dysfunction and delirium, indicate that current methods often lead to inappropriate dosing, particularly in vulnerable or elderly populations.

In recognizing the need for more accurate anesthetic drug administration, the present disclosure introduces a novel approach that stands in contrast to prior methods. In particular, systems and methods are provided that into account patient's individual brain function. Using various indicators, signatures and markers of brain function, or brain health, a patient's response may be determined and used to identify appropriate anesthesia or sedation. Indicators related to poor cognitive outcomes, such as a likelihood or probability of burst suppression, can also be determined and used to avoid, for example, overdosing.

As will become apparent from description herein, the provided systems and methods may be particularly beneficial for a variety of applications associated with medical procedures, including general anesthesia and sedation. For example, patients potentially at higher risk for poor cognitive outcomes, conditions or disorders may be pre-operatively identified using systems and methods described herein. In addition, information from signatures or markers of brain function may be used to give certain indications or treatments, such as specific regimens for anesthetic, post-anesthetic, or intensive care.

Use of electroencephalography (EEG) recordings to monitor and diagnose cognitive states in elderly patients has been previously demonstrated. For example, in one study, cortical gray matter was analyzed using both magnetic resonance imaging (MRI) and cortical EEG rhythms, in cognitively normal individuals, individuals with amnestic mild cognitive impairment (MCI) and Alzheimer's patients. Relative to the cognitively normal individuals, the MCI individuals displayed a decrease in the alpha-1 rhythm (8-10.5 Hz) source. Compared with the cognitively normal and the MCI individuals, the Alzheimer's disease patients had a decrease in the amplitude of the alpha-1 rhythm source and an increase in the amplitude of the delta rhythm (2-4 Hz) source. Overall, for the MCI and Alzheimer's disease patients, lower cortical gray matter volume and poor performance on cognitive tests were associated with lower alpha-1 and higher delta sources, suggesting that resting-state EEG measurements may provide ways of diagnosing impaired cognitive states.

Also, some studies have shown that the brain states of patients under general anesthesia may be tracked using the unprocessed EEGs and corresponding spectrograms. In addition, it was shown that differences likely exist between the unprocessed EEGs and spectrograms of cognitively normal elderly, MCI and Alzheimer's disease patients under general anesthesia. Similarly, observations of patients in the operating room showed that there are differences in EEG measurements between young, middle-aged and elderly patients under general anesthesia. For instance, a study by the inventors showed that anesthesia-induced frontal alpha waves diminish significantly with age. The generators of these frontal alpha waves overlap significantly with cortical regions that undergo profound neurodegeneration in aging and dementia. It is recognized herein that in addition to anesthesia-induced frontal alpha, pre-operative assessment of other EEG signal markers, such as posterior alpha, or assessments of cognitive function, could similarly be related to anesthetic dose response or drug requirements, as well as burst suppression, which is often associated with anesthetic overdose.

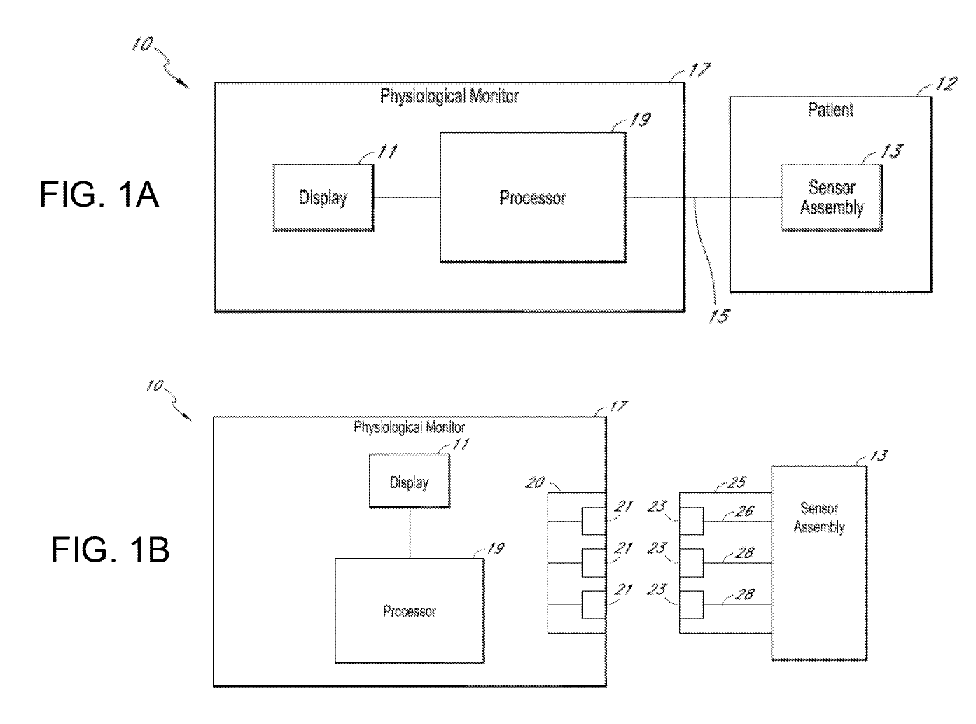

Referring now to the drawings, FIGS. 1A and 1B illustrate example patient monitoring systems that can be used to provide physiological monitoring of a patient, in accordance with aspects of the present disclosure. In general, these systems may include any device, apparatus or system configured for carrying out instructions for, and may operate as part of, or in collaboration with, various computers, systems, devices, machines, mainframes, networks or servers. In some aspects, the systems may be a portable. In this regard, the systems may be designed to integrate a variety of software and hardware capabilities and functionalities, and may be capable of operating autonomously or semi-autonomously.

For example, FIG. 1A shows an embodiment of a physiological monitoring system 10. In the physiological monitoring system 10, a medical patient 12 is monitored using a sensor assembly 13 having sensors that can transmit signals over a cable 15 or other communication link or medium to a physiological monitor 17. The physiological monitor 17 includes a processor 19 and, optionally, a display 11. The sensor assembly 13 may include various sensors or sensing elements such as, for example, electrical EEG sensors, blood pressure sensors, oxygenation sensors, respiration sensors, movement sensors, optical sensors, and so on. The sensors can acquire signals associated with one or more physiological parameters of the patient 12. The signals may then processed by one or more processors 19. The one or more processors 19 then communicate the processed signal to the display 11 if a display 11 is provided, or via another output. In some embodiments, sensors on the sensor assembly 13 may include various electrodes configured to detect electrophysiological signals. The electrodes may be connected to various amplifiers, filters, and other signal controlling electrical components.

In one embodiment, the display 11 may be incorporated in the physiological monitor 17. In another embodiment, the display 11 may be separate from the physiological monitor 17. In another embodiment, the monitoring system 10 may be a portable monitoring system. In yet another embodiment, the monitoring system 10 may be a pod, without a display, yet adapted to provide physiological parameter data to a display.

For clarity, a single block is used to illustrate the sensor assembly 13 shown in FIG. 1A. However, it should be understood that the sensor assembly 13 shown may include multiple assemblies, each configured with various sensors. In one embodiment, the sensor assembly 13 includes a single sensor of one of the types described below. In another embodiment, the sensor assembly 13 includes at least two EEG sensors. In still another embodiment, the sensor assembly 13 includes at least two EEG sensors and one or more brain oxygenation sensors, and the like. In each of the foregoing embodiments, additional sensors of different types may be optionally included. Other combinations of numbers and types of sensors are also suitable for use with the physiological monitoring system 10.

In some embodiments of the system shown in FIG. 1A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors in the sensor assembly 13.

In another embodiment, as shown in FIG. 1B, the sensor assembly 13 can include a cable 25 having three conductors within an electrical shielding. One conductor 26 can provide power to a physiological monitor 17, one conductor 28 can provide a ground signal to the physiological monitor 17, and one conductor 28 can transmit signals from sensors on the sensor assembly 13 to the physiological monitor 17. For multiple sensors, one or more additional cables 15 can be provided. In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 or couplings in the cable 25 can enable the cable to electrically connect to electrical interfaces 21 in a connector 20 of the physiological monitor 17. In another embodiment, the sensor 13 and the physiological monitor 17 communicate wirelessly.

In some configurations, although not explicitly shown in FIGS. 1A and 1B, systems according to the present disclosure may also include other elements including various input, output, and memory elements, as well as various communication networks for exchanging data and information between such various components.

Example input elements include a mouse, keyboard, touchpad, touch screen, buttons, and other user interfaces configured for receiving various selections, indications, and operational instructions from a user. Input elements may also include various drives and receptacles, such as flash-drives, USB drives, CD/DVD drives, and other computer-readable medium receptacles, for receiving various data and information. To this end, input elements may also include various communication ports and modules, such as Ethernet, Bluetooth, or WiFi, for exchanging data and information with various external computers, systems, devices, machines, mainframes, servers or networks.

In addition to being configured to carry out various steps for operating the system 10 of FIGS. 1A and 1B, the processor 19 may also be programmed to execute instructions stored in a non-transitory computer readable-media for example. In particular, the processor 19 may be programmed to analyze acquired and/or provided physiological data from a patient, including EEG data and cognitive data or cognitive testing information, to determine various indicators of brain function. Example of EEG data may include raw or processed signals or signal sets, waveforms, spectra or other EEG representations. Examples of cognitive data could include the results of cognitive tests such as the Mini-Mental State Exam (MMSE), or the Montreal Cognitive Assessment (MOCA). Other more comprehensive cognitive data might also be used, for instance, such as those that comprise the National Alzheimer's Coordinating Center's (NACC) Uniform Data Set (UDS) Neuropsychological Test Battery, including Digit Span Forward & Backward, Trail Making Test, Craft Paragraph Memory Test, Digit Symbol, Verbal Fluency, the Multilingual Naming Test, and so forth.

Various analysis methods may be used by the processor 19 to determine the various indicators, including waveform analyses, spectral analyses, frequency analyses, coherence analyses and so on. In some aspects, the processor 19 may be programmed to apply a multitaper technique to process the physiological data. In this manner, spectrogram or coherogram information or data may be generated. In addition, the processor 19 may be programmed to identify signatures or signal markers based on amplitudes, as well as other signal characteristics, of waveforms, power spectra, periodograms, spectrograms, and coherograms associated with or obtained from the physiological data. The processor 19 may also be programmed to analyze physiological data, such as EEG data, to determine a likelihood of a burst suppression.

Based on indicators of brain function, a response of a patient to the administration of drugs, or drug sensitivity, may be determined by the processor 19 and provided in a report. In doing so, the processor 19 may utilize reference information or data, as well as information or indications provided via an input or user interface. For example, the processor 19 may utilize accessed, provided or identified information regarding the patient, such as various patient characteristics, as well as information about drugs being administered. In some aspects, the processor 19 may be programmed to generate one or more pharmacodynamics curves based on the determined response. Based on the determined response, the processor 19 may then estimate a drug dose to achieve a predetermined or targeted state of anesthesia or sedation. In estimating the drug dose, the processor 19 may additionally or alternatively utilize the determined likelihood or probability of burst suppression. The processor 19 may control or communicate with a drug delivery system to deliver the estimated drug dose. To this end, the processor 19 may determine a drug infusion rate or a target drug concentration and relay information or instructions to a user or delivery system achieve them. In some aspects, the processor 19 may be programmed to operate the system 10 in a closed-loop to control the predetermined state of anesthesia or sedation of the patient.

Patient characteristics may be identified by the processor 19 by performing a comparison of the determined signal markers or signatures with those categorized in a reference, thus identifying a patient category closely resembling the patient-specific information. For example, a spectrogram or coherogram generated from the acquired data by the processor 19 may then be compared to a listing of spectrograms or coherograms to identify specific patient categories, related to patient characteristics, such as an apparent or likely patient age, or age range. In some implementations, a memory, database or other data storage location accessible by processor 19 may include reference information or other data. Such reference information can include a listing of patient categories, such as various age categories, along with associated signals, signal markers or signatures. For example, signal markers or signatures can include various signal amplitudes, phases, frequencies, power spectra, spectrograms, coherograms, and so forth. In some aspects, such reference information can be used by the processor 19, optionally including user input or selections, to determine specific patient characteristics, such an apparent or likely patient age, or other patient conditions or categories.

In some aspects, a data acquisition process may be regulated or modified based on selected and/or determined patient characteristics. For example, the processor 19 may be configured to determine and apply an appropriate scale during data acquisition using the patient characteristics, such as an apparent or likely patient age, identified in scout data. In other aspects, a display of acquired physiological data may be modified based on determined patient characteristics. Specifically, the data may be displayed against a scale determined by processor 19.

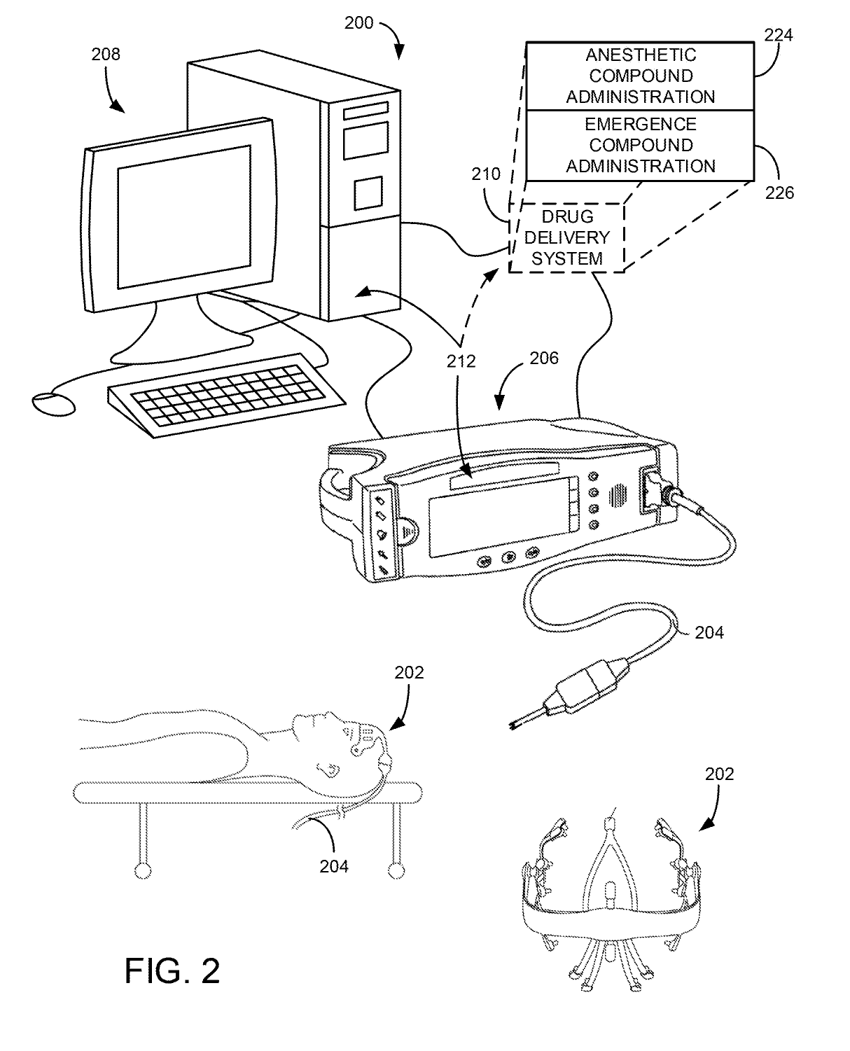

Specifically now referring to FIG. 2, an example system 200 in accordance with aspects of the present disclosure is illustrated. The system 200 may be constructed as a stand-alone brain monitoring device, or portable device, or could be incorporated as a central component of an existing brain monitoring device. As will be appreciated from forthcoming descriptions, the system 200 may find valuable usage within an operating room or an intensive care setting, in association with conducting a variety of medical procedures, such as during administration of an anesthetic, as well as within a pre- or post-operative evaluation situation.

The system 200 includes a patient monitoring device 202, such as a physiological monitoring device, illustrated in FIG. 2 as an electroencephalography (EEG) electrode array. However, it is contemplated that the patient monitoring device 202 may include a number of different sensors. In particular, the patient monitoring device 202 may also include mechanisms for monitoring galvanic skin response (GSR), for example, to measure arousal to external stimuli or other monitoring system such as cardiovascular monitors, including electrocardiographic and blood pressure monitors, and also ocular microtremor monitors. One realization of this design may utilize a frontal Laplacian EEG electrode layout with additional electrodes to measure GSR and/or ocular microtremor. Another realization of this design may incorporate a frontal array of electrodes that could be combined in post-processing to obtain any combination of electrodes found to optimally detect the EEG signatures described earlier, also with separate GSR electrodes. Another realization of this design may utilize a high-density layout sampling the entire scalp surface using between 64 to 256 sensors for the purpose of source localization, also with separate GSR electrodes. In some embodiments, electrodes in the electrode array may be connected to one or more amplifiers, filters, and other electronic components.

The patient monitoring device 202 is connected via a cable 204 to communicate with a monitoring system 206. Also, the cable 204 and similar connections can be replaced by wireless connections between components. The monitoring system 206 may be configured to receive raw or processed signals from patient monitoring device 202, such as signals acquired by the EEG electrode array, and assemble, process, and even display the signals in various forms, including time-series waveforms, spectrograms, and the like. In some modes of operation, the monitoring system 206 may be designed to acquire scout data, in the form of physiological or other data, from sensors on the patient monitoring device 202 and identify, using the scout data, signal markers, or signatures therein. For example, signal amplitudes, phases, frequencies, power spectra, and other signal markers or signatures, may be identified in scout data, and other acquired data, using various suitable methods. In addition, a multitaper technique may be performed for identifying and accounting for a dynamic range of signals spanning several orders of magnitude. Such signal markers or signature may then be used by the monitoring system 206 to determine various patient characteristics, including an apparent and/or likely patient age, as well as a response or sensitivity to administered drugs.

In one embodiment, acquisition of physiological data using monitoring system 206 may be adjusted or regulated based patient characteristics determined from scout data. Specifically, the monitoring system 206 may be configured to determine a scale consistent with certain determined patient characteristics, and adjust subsequent data acquisition, based on the determined scale and/or any indication provided by user. For instance, data acquisition may be regulated by adjusting one or more amplifier gains on various sensors, along with other data acquisition parameters. Moreover, in some aspects, the monitoring system 206 may be further configured to format various acquired physiological data to be displayed against the scale. In this manner, an age-appropriate scale may be determined based on the apparent and/or likely patient age, and any subsequent data acquisition using a selected age-appropriate scale would generate and illustrate age-compensated data.

As illustrated, the monitoring system 206 may be further connected to a dedicated analysis system 208. However, the monitoring system 206 and analysis system 208 may be integrated or combined into a common system. The analysis system 208 may receive EEG waveforms from the monitoring system 206 and, as will be described, analyze the EEG waveforms and signatures therein. However, it is also contemplated that any analysis or processing functions of the monitoring system 206 and analysis system 208 may be shared or individually distributed, as required or desired.

In some aspects, information related to determined characteristics of a patient undergoing a specific medical procedure may be provided to a clinician or operator of system 200. For example, it was previously found that elderly patients were more likely to enter burst suppression in the operating room. Specifically, burst suppression is the profound state of brain inactivation in which bursts of electrical activity are interspersed with isoelectric periods termed suppressions. Brain states of anesthetic-induced unconsciousness, defined by the alpha wave (8-10 Hz) and slow wave (0.1-4 Hz) signal oscillations, can be obtained with doses of anesthetics that are less than those required to produce burst suppression. This may mean reducing anesthetic dosing to levels substantially less than what are currently recommended for elderly individuals. Because currently recommended doses typically place elderly patients into burst suppression, adequate states of general anesthesia and reduced anesthetic exposure may be achievable by titrating anesthetic dosing based on real-time EEG monitoring. Hence system 200 may provide, based on determined patient characteristics, information for use in selecting an appropriate anesthetic dosing. In this manner, for example, incidence of post-operative cognitive disorders for elderly patients under general anesthesia may be reduced.

In another example, monitoring system 206 and/or analysis system 208 may be capable of providing a pre- or post-operative assessment of specific patients, such as the young, middle-aged and elderly, as well as drug addicted patients, to determine prior information that could be used to identify and/or predict specific patient conditions, including anesthetic sensitivity, and any potential for post-operative complications, such as cognitive disorders. Moreover, specific regimens for anesthetic care, post-anesthesia care, or intensive care, may also be provided.

The system 200 may also include a drug delivery system 210. The drug delivery system 210 may be coupled to the analysis system 208 and monitoring system 208, such that the system 200 forms a closed-loop monitoring and control system. Such a closed-loop monitoring and control system in accordance with the present disclosure is capable of a wide range of operation, but includes user interfaces 212 to allow a user to configure the closed-loop monitoring and control system, receive feedback from the closed-loop monitoring and control system, and, if needed reconfigure and/or override the closed-loop monitoring and control system. In some configurations, the drug delivery system 210 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness. In some aspects, a determined response or drug sensitivity of a patient, as described, may be utilized by the drug delivery system 210, in a closed-loop fashion to achieve a predetermined or targeted state of anesthesia or sedation.

For example, methylphenidate (MPH) can be used as an inhibitor of dopamine and norepinephrine reuptake transporters and actively induces emergence from isoflurane general anesthesia. MPH can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. Also, ethylphenidate or other agents can be used to actively induce emergence from isoflurane, propofol, or other general anesthesia by increasing arousal using a control system, such as described above.

Therefore, a system, such as described above with respect to FIG. 2, can be provided to carry out active emergence from anesthesia by including a drug delivery system 210 with two specific sub-systems. As such, the drug delivery system 210 may include an anesthetic compound administration system 224 that is designed to deliver doses of one or more anesthetic compounds to a subject and may also include a emergence compound administration system 226 that is designed to deliver doses of one or more compounds that will reverse general anesthesia or the enhance the natural emergence of a subject from anesthesia.

For example, MPH and analogues and derivatives thereof induces emergence of a subject from anesthesia-induced unconsciousness by increasing arousal and respiratory drive. Thus, the emergence compound administration system 326 can be used to deliver MPH, amphetamine, modafinil, amantadine, or caffeine to reverse general anesthetic-induced unconsciousness and respiratory depression at the end of surgery. The MPH may be dextro-methylphenidate (D-MPH), racemic methylphenidate, or leva-methylphenidate (L-MPH), or may be compositions in equal or different ratios, such as about 50 percent:50 percent, or about 60 percent:40 percent, or about 70 percent:30 percent, or 80 percent:20 percent, 90 percent: 10 percent, 95 percent:5 percent and the like. Other agents may be administered as a higher dose of methylphenidate than the dose used for the treatment of Attention Deficit Disorder (ADD) or Attention Deficit Hyperactivity Disorder (ADHD), such as a dose of methylphenidate can be between about 10 mg/kg and about 5 mg/kg, and any integer between about 5 mg/kg and 10 mg/kg. In some situations, the dose is between about 7 mg/kg and about 0.1 mg/kg, or between about 5 mg/kg and about 0.5 mg/kg. Other agents may include those that are inhaled.

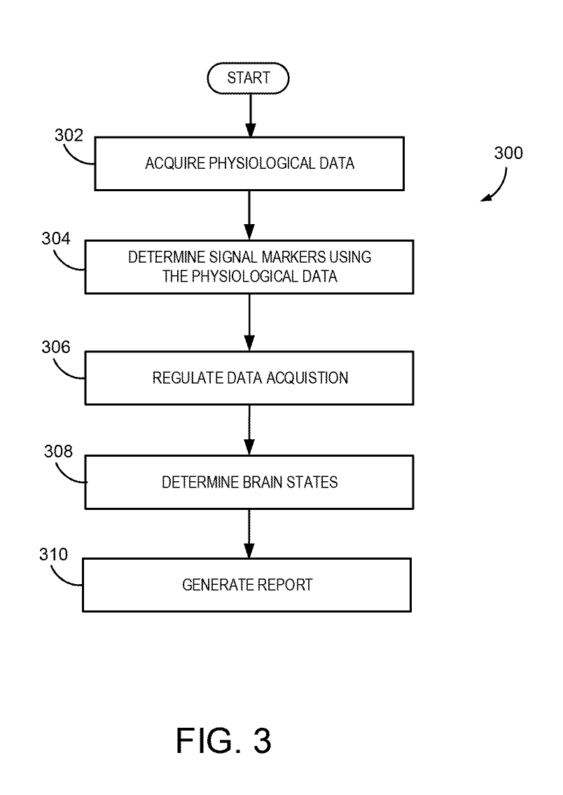

Turning to FIG. 3, a process 300 in accordance with aspects of the present disclosure is shown. The process 300 may be carried out using any suitable device, apparatus or system, including the systems described with reference to FIGS. 1A, 1B and 2. In some aspects, the process 300 may be implemented as a program or executable instructions stored in non-transitory computer readable media.

Beginning with process block 302, any amount of physiological data may be acquired. The physiological data is representative of physiological signals, such as EEG signals, obtained from a patient using, for example, the patient monitoring device 202. In some aspects, the physiological data may include scout data for purposes that include determining various patient characteristics. Then at process block 304, signal markers or signatures are identified or determined using the acquired physiological data. For example, signal amplitudes, phases, frequencies, power spectra, and other signal markers or signatures, may be identified in scout data, and/or other acquired data, using various suitable methods.

In some preferred embodiments, the signal markers or signatures may be used to determine patient characteristics, including an apparent and/or likely patient age, as well a drug response or drug sensitivity. In addition, process block 304 may also include steps of determining a scale consistent with determined patient characteristics. In one aspect, spectral estimation methods, such as the multitaper method, may be employed to account for a wide dynamic range of signals spanning many orders of magnitude. In another aspect, an automatic estimation of signal amplitudes may be performed to infer a correct age cohort and attendant settings for a visualization scale, as well as for acquisition amplifier gains.

At the next process block 306, using the signal markers or signatures determined from the scout data, a data acquisition process may be adjusted or regulated. For instance, data acquisition may be regulated by adjusting one or more amplifier gains, along with other data acquisition parameters. In some aspects, regulating data acquisition may also include determining and using a scale consistent with determined patient characteristics, and adjusting a subsequent data acquisition process based on the determined scale and/or any indication provided by user. By way of example, an age-appropriate scale determined at process block 304, based on the apparent and/or likely patient age, may be used, and any subsequent data acquisition using a selected age-appropriate scale would generate age-compensated data. In other aspects, a display of physiological data acquired at process block 302 may be modified using the scale.

At process block 308, data acquired in a manner described may be used to determine current or future brain states of patient. For example, analyzed or processed EEG waveforms assembled using age-compensated data may be used to assess a present and/or future depth of anesthesia or sedation. In addition, determining such brain states may also include any information provided by a clinician or user, such as information related to a medical procedure.

Then at process block 310 a report is generated, for example, in the form a printed report or a real-time display. The report may include raw or processed data, signature information, indications of current or future brain states, as well as information related to patient-specific characteristics, including as a likely and/or apparent patient age. Displayed signature information or determined states may be in the form of a waveforms, spectrograms, coherograms, probability curves and so forth. In some aspects, the report may include formatted physiological data displayed against a scale. In other aspects, the report may indicate an anesthetic sensitivity, a probability for post-operative complications, such as cognitive disorders, and also regimens for anesthetic care, post-anesthesia care, or intensive care, and so forth.

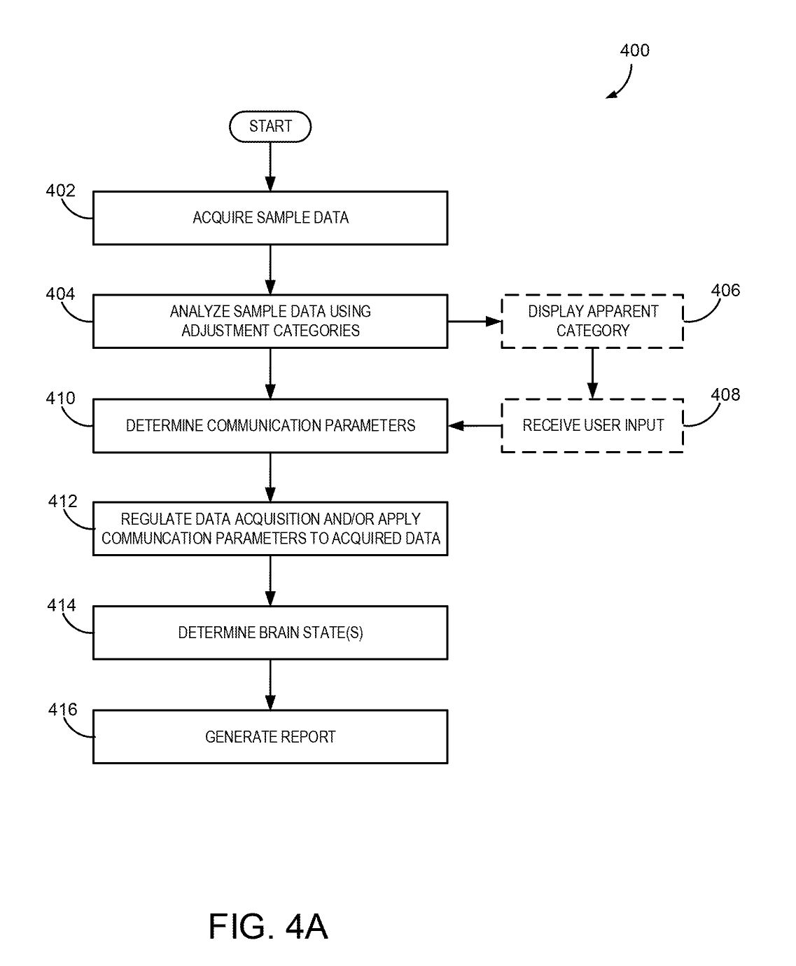

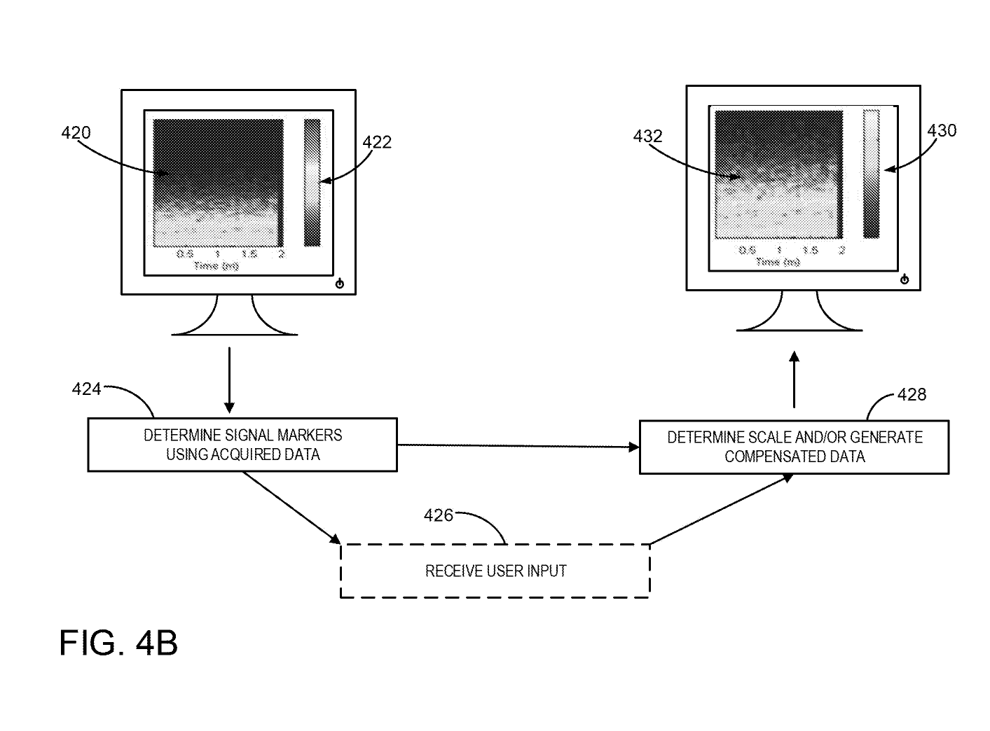

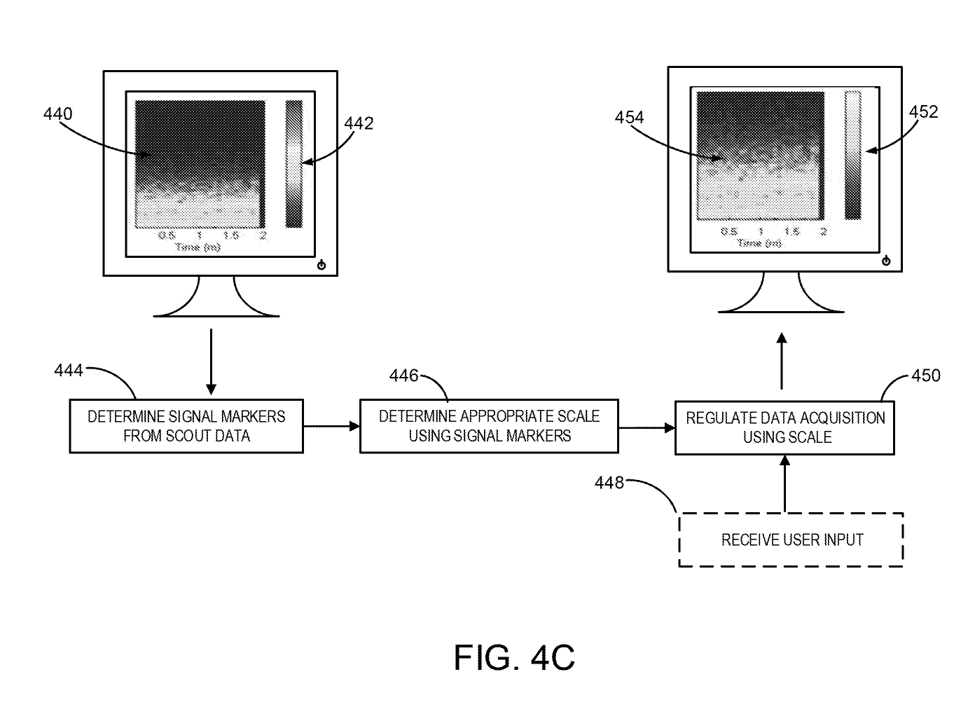

Turning to FIG. 4A, steps of another process 400 in accordance with aspects of the present disclosure are illustrated. Similarly, the process 400 may be carried out using any suitable device, apparatus or system, including the systems described with reference to FIGS. 1 and 2. The process 400 may also be implemented as a program or executable instructions stored in non-transitory computer readable media.

Specifically, the process 400 begins at process block 402 where sample or scout data is acquired using, for example, patient monitoring systems, as described. At process block 404, the sample data is then analyzed using various adjustment or reference categories, to identify patient categories representative of the acquired sample data. Specifically, this step includes identifying signal markers or signatures in the sample data and performing a comparison with signal markers or signatures associated with the reference categories. For example, signal amplitudes, phases, frequencies, power spectra, coherences, and other signal markers or signatures, can be detected in the sample data using various suitable methods.

Analysis of the sample data, as performed at process block 404, can indicate specific patient characteristics, including an apparent and/or likely patient age as well as a drug response or drug sensitivity. In some aspects, an identified or apparent category indicating specific patient characteristics may be optionally displayed at process block 406. Moreover, at process block 408 a user input may also be received.

Subsequently, at process block 410 a determination is made with respect to various communication parameters. This includes taking into consideration determined or inferred patient characteristics or categories, and optionally a user input. For example, an age-appropriate scale for the acquired data may be determined at process block 410 based on determined patient characteristics and/or signals, signal markers or signatures present in the acquired data. Then at process block 412, a subsequent data acquisition may be regulated using the determined communication parameters to acquire age-appropriate data. As described, regulating data acquisition may include appropriately adjusting or modifying various amplifier gains using the communication parameters. In some aspects, the determined communication parameters may be directly applied to the acquired sample data. For example, an age-appropriate scale may be applied to the sample data to create age-appropriate or compensated data.

Then, at process block 414, data acquired or processed in a manner described may be used to determine current or future brain states of patient. For example, analyzed or processed EEG waveforms assembled using age-compensated data may be used to assess a present and/or future depth of anesthesia or sedation. In addition, determining such brain states may also include any information provided by a clinician or user, such as information related to a medical procedure.

Then at process block 416 a report is generated of any suitable shape or form. In some aspects, the report may be a display scaled data or data categories describing the data. In other aspects, the report may indicate an anesthetic sensitivity, a probability for operative or post-operative complications, an apparent or likely patient age, and other information related to aspects of the present disclosure.

Turning to FIG. 4B a schematic diagram illustrating steps in accordance with one embodiment of the present invention is shown. Specifically, acquired data 420, optionally processed and displayed using a raw scale 422, may be used to determine signal markers or signatures at step 424. As described, this step may include a number of processing or analysis steps, including waveform analyses, spectral analyses, frequency analyses, coherence analyses and so on. Then at step 428 information related to the determined signal markers or signatures may be determined. Particularly, patient characteristics may be identified by performing a comparison of the determined signal markers or signatures with those categorized in a reference, thus identifying a most similar patient category. For example, an apparent or likely age may be identified.

In this manner, using information identified in the acquired data 420, an appropriate scale 430 for the acquired data 420 may be determined and/or selected at step 428 and applied to the acquired data to generate and display scaled or modified data 432. For example, a determined apparent or likely patient age, or age range, may be used to identify an age-appropriate scale, and generate age-compensated data, which may be optionally displayed. It may be appreciated that the appropriate scale 430 may indicate a wider or a narrower dynamic range as compared to the raw scale 422.

In some aspects, this step may also include receiving a user input as indicated by step 426. For example, a clinician may provide information relevant to a monitored patient, including a patient's real age, as well as information related to a medical procedure, such as a specific anesthetic or dose. In some aspects, the modified data 432, appropriately scaled and/or displayed, may then be utilized in a brain analysis process to correctly identify brain states of the patient.

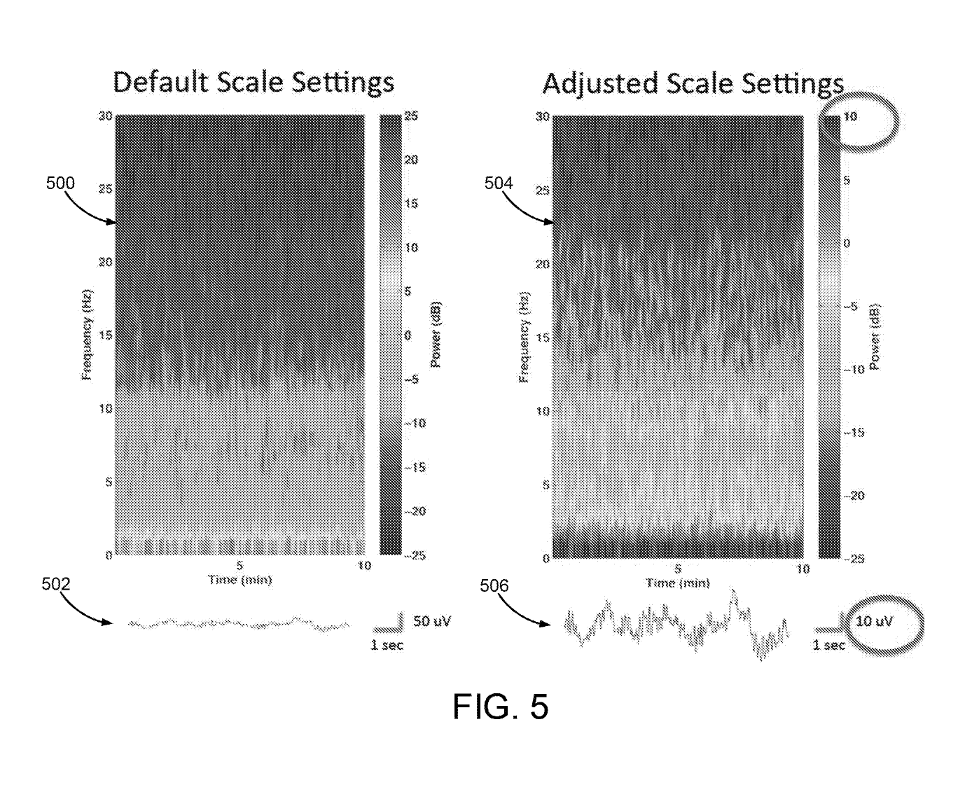

By way of example, FIG. 5 shows an example scale adjustment for a 61 year old patient, in accordance with aspects of the present disclosure. Specifically, acquired waveform data 500 and spectrogram data 502 is displayed using default scale settings, which may not be ideal or appropriate. Following steps, as described, scaled spectrogram data 504 and scaled waveform data 506 may generated and displayed against an appropriate scale determined based on identified patient characteristics, such as likely or apparent age. As appreciated from this example, appropriate scaling may help depict additional structure that would not be visible otherwise.

Turning to FIG. 4C a schematic diagram, illustrating steps in accordance with another embodiment of the present disclosure, is shown. Using analyses as described, scout data 440 may be utilized at step 444 to determine signal markers present therein. The scout data 440 may be optionally displayed against a raw scale 442, as shown. At step 446 an appropriate or compensated scale 452, which may different than a default scale setting, is determined using signal markers and other information determined from the scout data 440. Optionally other parameters may also be determined at step 446 from the scout data 400, including a number of data acquisition parameters. For example, appropriate amplifier gains may also be identified via dynamic range exhibited by the scout data.

At step 450 a data acquisition process 450 may be regulated or modified using the appropriate scale 452 in order to generate appropriate data 454. Optionally, this step may include a user input that is received, as indicated by step 448. For example, a clinician may provide information relevant to a monitored patient, including a patient's real age, as well as information related to a medical procedure, such as a specific anesthetic or dose. Additionally, a user input may include selection of acquisition parameters, over-riding instructions, or other input related to the data acquisition process. In some aspects, the appropriate data 454, suitably acquired and/or displayed using patient-specific characteristics, may then be utilized in a brain analysis process to correctly identify brain states of the patient.

Examples of acquired data, scout data, and modified data, shown in FIGS. 4B, 4C and 5 as spectrograms, or portions thereof, are given for illustrative purposes, and are in no way limiting. That is, it may be understood that other types of data may be utilized, processed, displayed, or scaled, including waveform data, spectral data, coherogram data, and so forth.

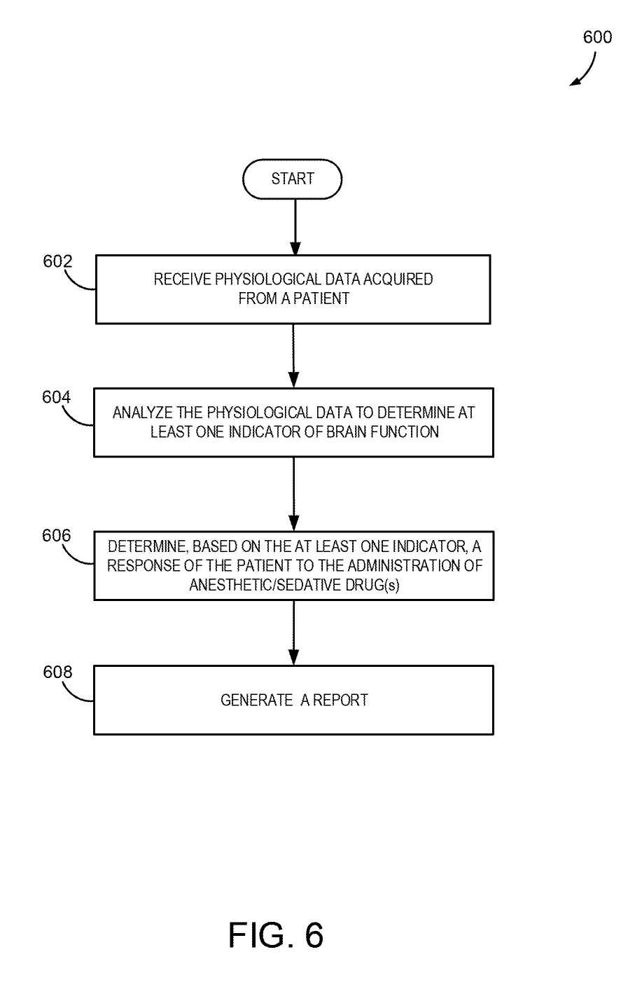

Turning now to FIG. 6, a diagram setting forth steps of another process 600, in accordance with aspects of the present disclosure, is shown. Similarly, the process 600 may be carried out using any suitable device, apparatus or system, including the systems described with reference to FIGS. 1A, 1B and 2. The process 600 may also be implemented as a program or executable instructions stored in non-transitory computer readable media.

The process 600 may begin at process block 602 with receiving physiological data, such as EEG and cognition data, acquired from a patient. In some aspects, a data acquisition may also be performed at process block 602. Then, an analysis of the received or acquired data may be performed at process block 604 to determine at least one indicator of brain function. As described, various analysis techniques may be applied to determine the at least one indicator including waveform analyses, spectral analyses, frequency analyses, coherence analyses and so on. In some aspects, a multitaper technique may be applied. In addition, signatures or signal markers may be determined based on amplitudes in waveforms or power spectra generated from the physiological data. In some aspects, a probability or likelihood of a burst suppression may be determined in the analysis performed at process block 604 using the physiological data. Furthermore, a data acquisition may be adapted at process block 602, based on the various analyses carried out and information obtained therefrom, such as a determined likely or apparent age, as described.

Based on indicators of brain function, a response of the patient to the administration of drugs, or a drug sensitivity, may be determined as indicated by process block 606. In determining the response, reference information or data, as well as information or indications provided via an input or user interface, may be utilized. For example, accessed, provided or identified information regarding the patient, such as various patient characteristics, as well as information about drugs being administered, may be utilized to determine the response. Example patient characteristics include a patient's calendar age, likely or apparent age, height, weight, gender, medical condition, and so forth. Medical conditions could include prior diagnoses of cognitive impairment, dementia, or neurovascular disease, for instance, which might influence the brain's response to anesthetic or sedative drugs. In general, in addition to information from anesthesia-induced EEG measurements, any prior information obtained from the patient that would indicate brain condition, brain age or brain health may be used to determine the response.

In some aspects, one or more pharmacodynamics curves may be generated at process block 606. Based on the determined response, a drug dose suitable to achieve a predetermined or targeted state of anesthesia or sedation may be estimated. In estimating the drug dose, the determined likelihood or probability of burst suppression may be additionally or alternatively utilized. Using the estimated drug dose, an infusion rate or a target drug concentration may also be determined for controlling an anesthetic state of the patient.

A report may then be generated, as indicated by process block 608. The report may be in any form and include any information. In particular, the report may indicate a current and/or future anesthesia or sedation states of the patient, as well as other patient information, including various patient characteristics and drug sensitivities. For instance, the report may also indicate a probability of burst suppression. The report may further indicate estimated drug doses, drug infusion rates or target drug concentrations. In some aspects, the report may be directed to a display or to a drug delivery system, providing information and operational instructions to control the drug delivery system in order to achieve targeted or predetermined anesthesia or sedation states for the patient.

The above-described systems and methods may be further understood by way of example. This example is offered for illustrative purposes only, and is not intended to limit the scope of the present invention in any way. Indeed, various modifications in addition to those shown and described herein will become apparent to those skilled in the art from the foregoing description and the following examples and fall within the scope of the appended claims. For example, specific examples of brain states, medical conditions, levels of anesthesia or sedation and so on, in association with specific drugs and medical procedures are provided, although it will be appreciated that other drugs, doses, states, conditions and procedures, may be considered within the scope of the present invention. Furthermore, examples are given with respect to specific indicators related to brain states, although it may be understood that other indicators and combinations thereof may also be considered within the scope of the present invention. Likewise, specific process parameters and methods are recited that may be altered or varied based on variables such as signal amplitude, phase, frequency, duration and so forth.

### EXAMPLE

As described, there are a number of ways in which markers of brain function could be used to estimate or predict the dose response of patients receiving general anesthetic and sedative drugs. As described, physiological data, such as the EEG, and/or cognitive testing information, could be analyzed to determine a patient's anesthetic or sedative dose response or sensitivity information. Such information could be represented in different ways, including in the form of a predicted or estimated pharmacodynamic response curve, or in terms of a likelihood of burst suppression. Physiological data could also be analyzed to estimate drug infusion rates or drug target concentrations, or ranges thereof, sufficient to achieve unconsciousness for general anesthesia, or sedation, or brain states generally associated with anesthesia overdose, such as burst suppression states.

In the analysis, various quantitative or computational representations of the relationship between anesthesia-induced signatures and different dose-response characteristics, including the likelihood of burst suppression, could be used. Such quantitative or computational representations could be stored in a database, for example, in the form of mathematical or statistical models relating EEG data features, cognitive data features, and other markers of brain function to desired variables, such as dose response curves, pharmacological models, infusion rates or target concentrations and ranges, likelihoods or probabilities of burst suppression, and so on.

The database or models therein could also include pertinent covariate information for interpreting the EEG, cognitive testing results, and other markers of brain function, including patient variables and history such as calendar age, height, weight, or gender, as well as information about the drugs administered to the patient, their doses and timing. Covariate information could also include a patient's history of neurological or cognitive conditions, such as cognitive impairment, dementia, or Alzheimer's disease, for instance. The representation of the EEG in this database or model could be made in any number of ways, including frequency-dependent measures such as spectrum, coherence, spectrogram, or cohereogram, time-domain measures such as amplitude or morphology, or other measures such as cross-frequency coupling, for instance. Inferences from the database or model could be made using any number of appropriate established methods, including look-up tables, prediction using a regression or statistical model, perhaps employing Bayesian inference to jointly incorporate age and EEG-related information, machine learning methods, or through cross-correlation, clustering, or related techniques.

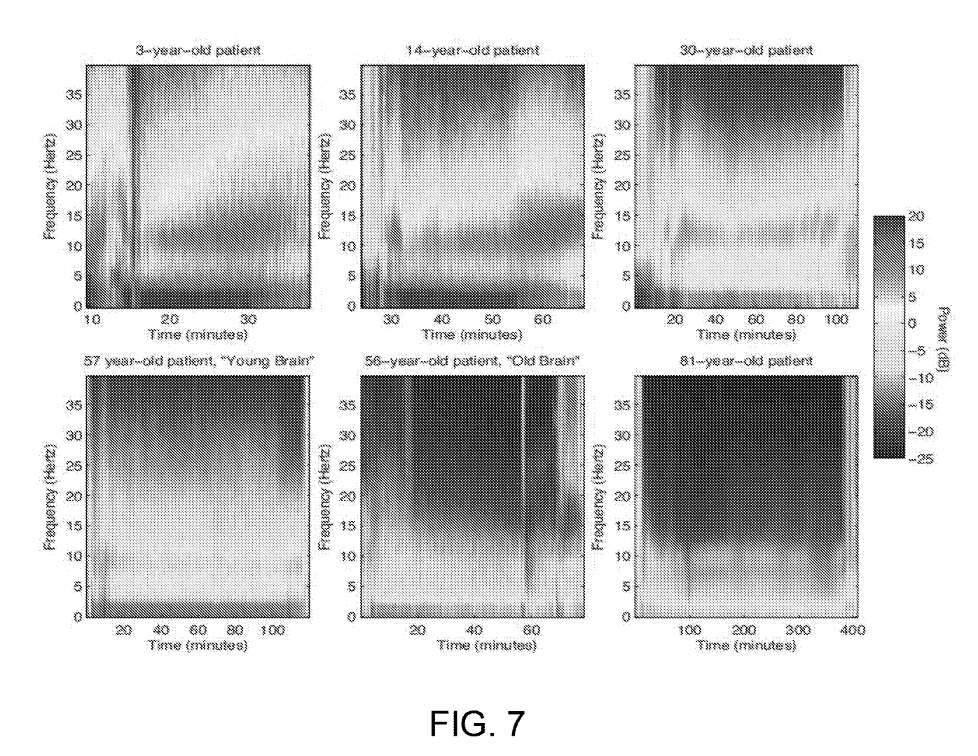

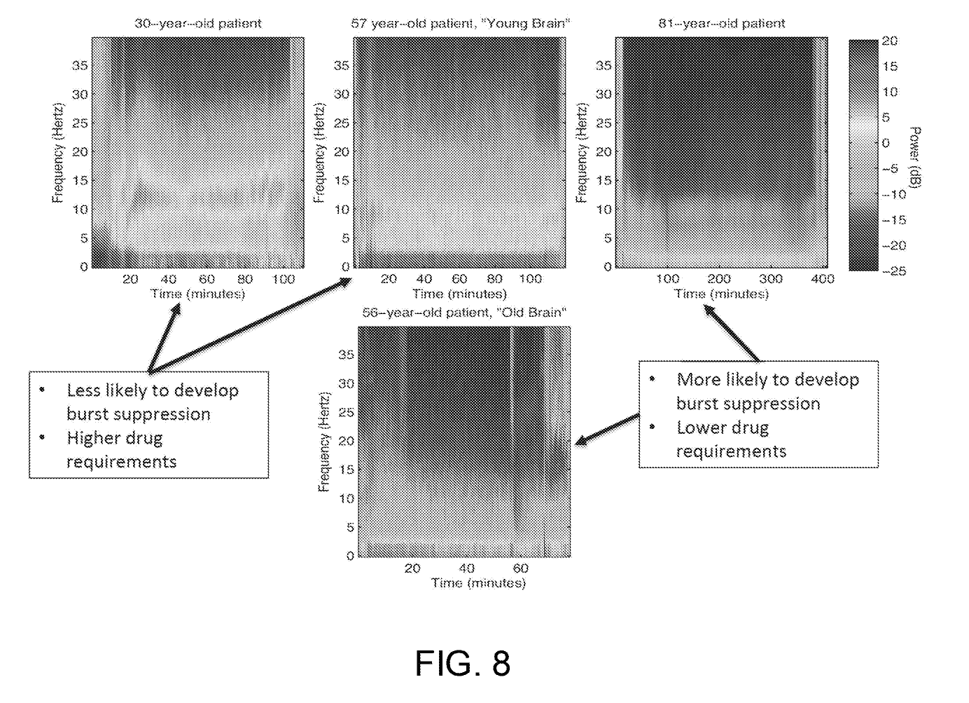

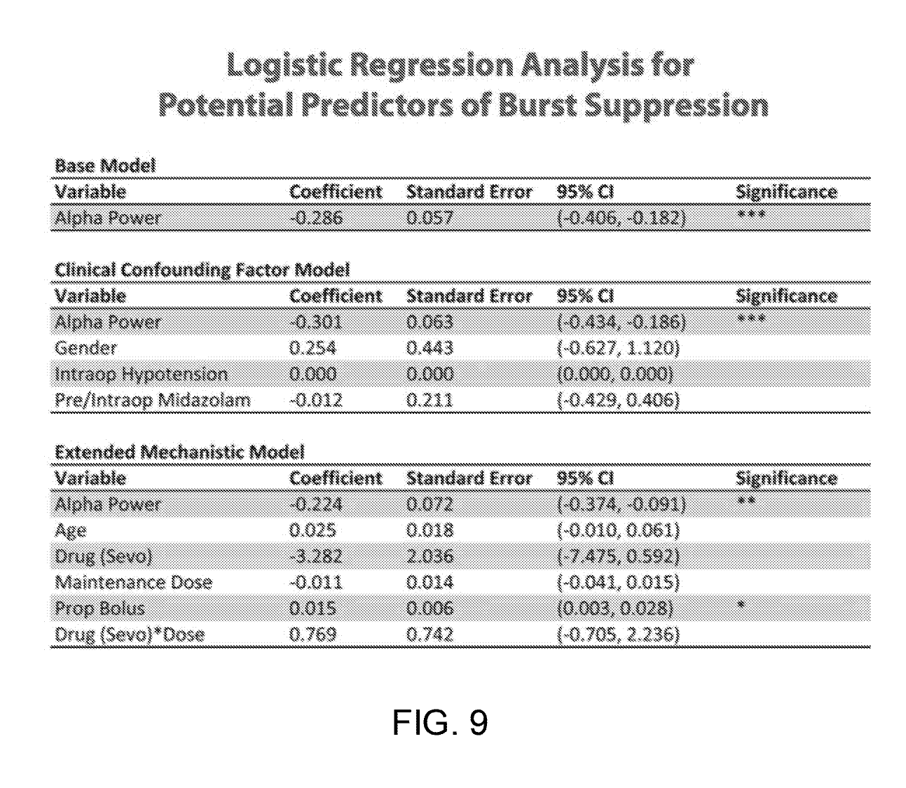

To illustrate the relationship between anesthesia-induced EEG and burst suppression, 4-lead EEG data was recorded during routine care of patients receiving general anesthesia across a broad range of patient ages, from 18 to 89 years of age. Data were recorded using two different drugs, propofol and sevoflurane, two of the most commonly used anesthetic drugs. Data from 155 patients who received propofol (n=60) or sevoflurane (n=95) as the primary anesthetic was analyzed. The EEG spectra and coherence from a 2 min period of stable anesthetic maintenance was estimated. A multitaper spectral analysis was used to estimate alpha power from the EEG recordings. FIGS. 7 and 8 show example spectrograms for representative patients. Burst suppression was operationally defined by the presence of at least three consecutive suppression events within a 1 min period occurring within a window beginning 10 minutes after induction through the end of the procedure. A logistic regression analysis was performed to characterize the effects of different variables, including age, anesthetic dose (propofol infusion rate and sevoflurane age-adjusted minimum alveolar concentration), and EEG alpha power on the probability of an episode of burst suppression. FIG. 9 illustrates the structure and analysis of different configurations of such variables in the logistic regression analysis. In all models, alpha power was shown to be a strong predictor of burst suppression.

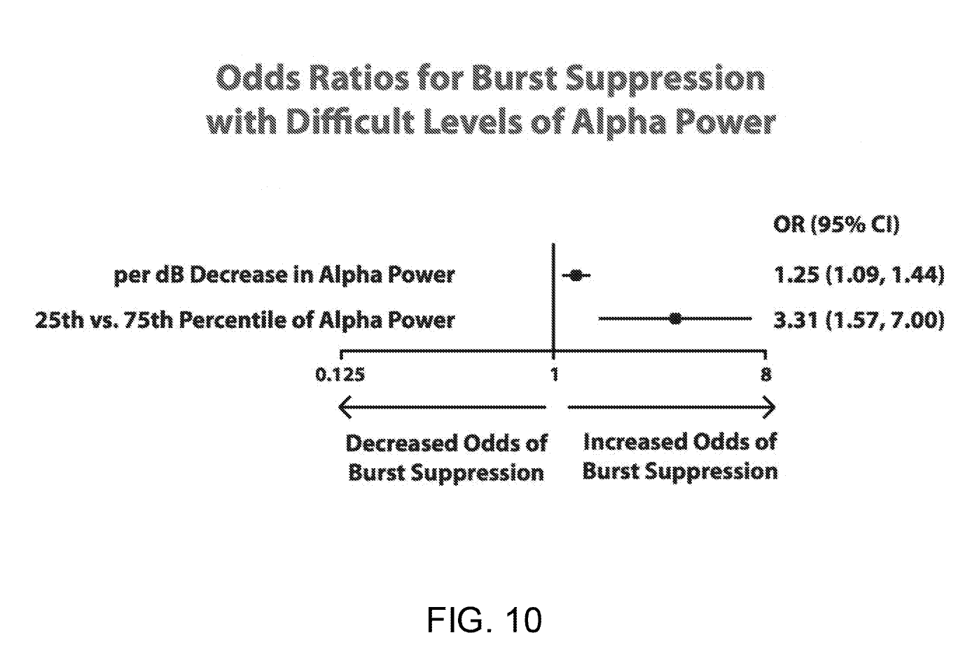

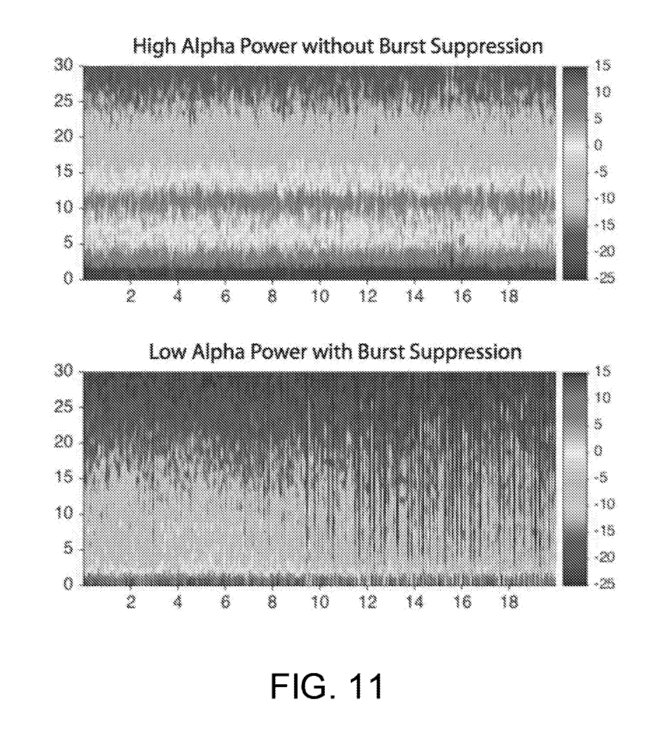

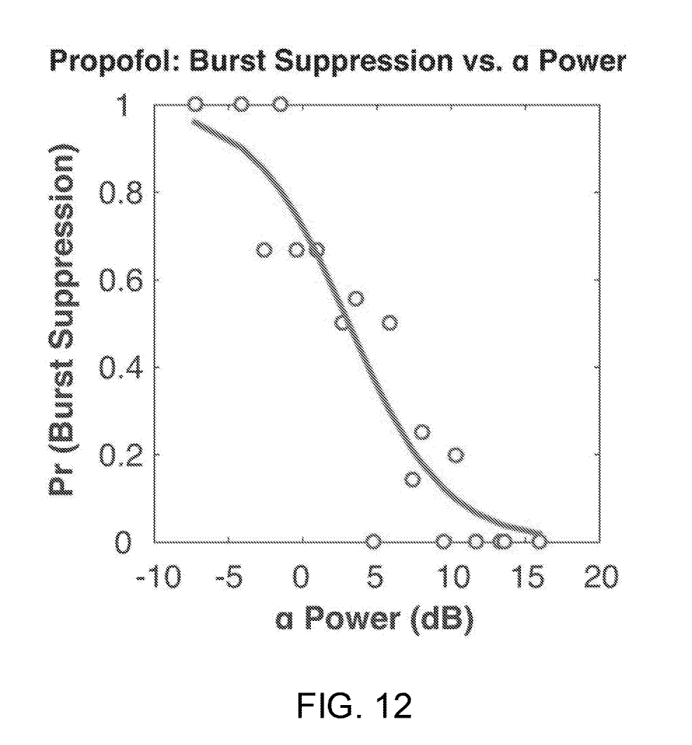

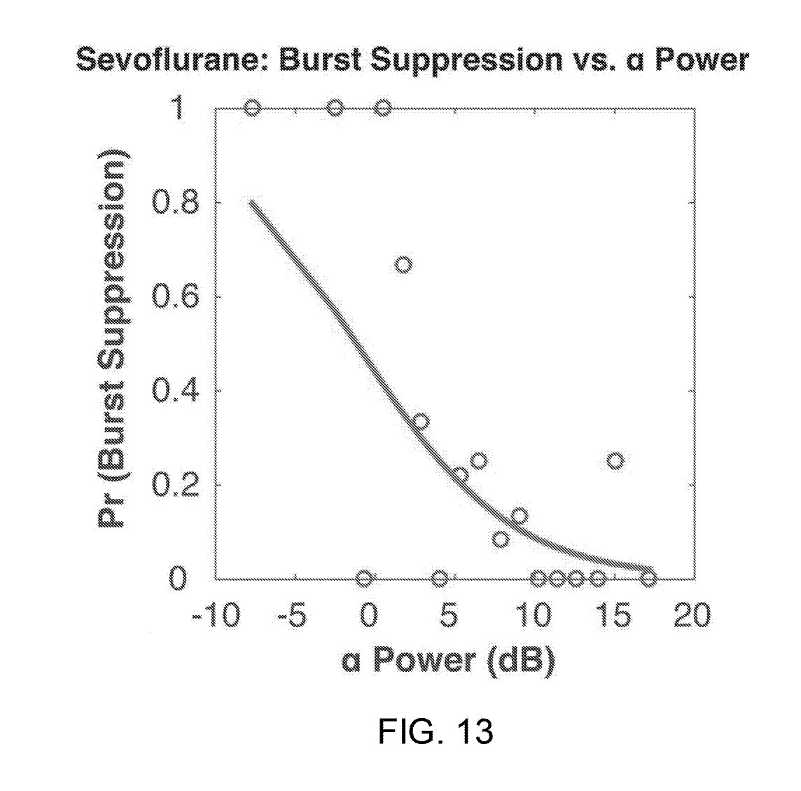

As shown in FIGS. 11, 12 and 13, alpha power alone may be used as an indicator to accurately predict burst suppression under propofol and sevoflurane, respectively. FIG. 10 illustrates how the relationship between alpha power and burst suppression can be represented as an odds ratio. The fact that the likelihood or odds of burst suppression increases as alpha power decreases indicates that alpha power may be a marker of anesthetic drug sensitivity or drug requirements. In particular, patients with low alpha power require lower amounts of anesthetic drugs to maintain a state of unconsciousness. In addition, these patients are more likely to be over-dosed at lower doses of anesthetic drugs. Thus, alpha power can be used to predict anesthetic drug requirements, drug sensitivity, and likelihood of burst suppression.

Given that anesthesia-induced alpha waves have a basic neurophysiological mechanism that is similar to other EEG oscillations, including awake posterior alpha waves and sleep spindles, other related EEG oscillations recorded pre-operatively could also be used to predict anesthetic drug requirements and likelihood of burst suppression. Moreover, because alpha waves are related to cognition, cognitive testing could also serve as a means to predict anesthetic drug requirements and likelihood of burst suppression.

In contrast to the multitaper method, a more commonly used method of spectral estimation is the periodogram, which uses the discrete Fourier transform as the basis function of spectral decomposition. For a random signal xₖ sampled at intervals of Δt where k=0, . . . , N−1, Sₚ(f), the periodogram at frequency f is defined as

$$\text{?} ( f ) = Δ t {\vert \text{?} \text{?} \vert}^{2} . \\ \text{?} \text{indicates text missing or illegible when filed}$$

Because of the finite duration of experimentally observed signals, the periodogram suffers from two potential problems. First, the spectral estimate is biased—i.e., on average, the periodogram will be different from the true underlying spectrum. The consequence of this bias is that peaks within the spectrum can appear less distinct and blurred across frequencies. In addition, the periodogram has high variance, due to the fact that the data is a single realization of a random signal. This produces noisy estimates of the spectrum.

In an effort to reduce the estimate bias, a common technique is to apply a taper or window to the data. Common tapers used are Welch, Hanning, and Hamming functions, which tend to limit the amount of bias or blurring. S [symbol] (f), the single-tapered periodogram at frequency f is defined as

$$\text{?} ( f ) = Δ t {\vert \text{?} \text{?} \vert}^{2} , \\ \text{?} \text{indicates text missing or illegible when filed}$$

where wₖ is the value of the taper at time k. While the single-tapered spectral estimate reduces the estimation bias as compared to the periodogram, the commonly used tapers are not necessarily optimal for bias reduction and the variance of the spectral estimate is still high.

Therefore, a multitaper spectral estimation was designed to improve on the single-taper estimator by simultaneously addressing the issues of bias and variance. Specifically, this was achieved by averaging the estimates from multiple tapers applied to the same data window, which were optimized to limit bias. These tapers were taken from a class of functions called the discrete prolate spheroid sequence (DPSS), also known as the Slepian sequence. These functions are designed to optimize the concentration of power in the main lobe with respect to the rest of the function, such that, for a taper W

$${max}_{w} \vert \frac{power in the main lobe}{total power} \vert .$$

This is referred to as solving the spectral concentration problem. Optimizing for main lobe power concentration (which involves eigenfunctions) produced tapers that were orthogonal, meaning they each extract independent estimates of the spectrum from the same window of data. In doing so, multiple estimates with reduced biased can be averaged together to produce a single estimate of the spectrum with reduced bias and variance.

The choice of L=|2TW|−1 for the number of tapers was based on the fact that the significance of adding a taper drops precipitously when the number of tapers reaches a quantity known as the Shannon number, which in this case happens to be equal to 2TW. Thus, by setting the number of tapers to one less than this quantity, an efficient estimate can be produced that uses no more than the maximum number of significant tapers. It is noted that TW is technically half the time-bandwidth product (or time-half-bandwidth product), however the term time-bandwidth product is used herein for simplicity.

Given a set of L DPSS tapers (w^{L}, . . . , w^{L}), S [symbol] (f), the multitaper spectral estimate at frequency f may be defined as

$$\text{?} ( f ) = \frac{1}{L} \text{?} {\vert \text{?} \text{?} {x}_{k} \text{?} \vert}^{2} , \\ \text{?} \text{indicates text missing or illegible when filed}$$

in which the spectral estimate is the average of the single-taper estimates for each taper. It can be shown that the multitaper estimate reduces the variance by a factor of approximately L compared to single-tapered estimates.

Referring specifically to FIG. 7 shows example EEG spectrograms during propofol general anesthesia for patients across a wide range of ages from childhood through old age. The spectrogram for the 30-year-old patient in this example shows characteristic slow (<1 Hz) and alpha (8-12 Hz) oscillations consistent with the unconscious state during propofol anesthesia. In the 57-year-old patient, the same pattern is visible, but in the 81 year old patient, this pattern is faint and difficult to discern because the EEG signal and EEG power are much smaller. With increasing age, patients may experience different rates of aging and cognitive decline. FIG. 7 shows a 56 year old patient whose EEG spectrogram more closely resembles that of the 81-year old patient than the 57 year old patient who is closer in chronological age. This could reflect a higher degree of apparent aging in the 56 year old patient. As shown, from childhood through old age, EEG power and EEG signal amplitudes can decrease by an order of magnitude.

Beyond the age-dependent decline in slow and alpha oscillations, there are individual differences in alpha power that can be observed across different patients, as illustrated in FIG. 8. For instance, as shown, the 30-year-old and 57-year old “young brain” patient are less likely to develop burst suppression and would likely have higher drug requirements. On the other hand, the 56-year-old “old brain” patient and the 81-year-old patient are more likely to develop burst suppression and hence would likely have lower drug requirements. As appreciated from this data, given an estimate of alpha power from the EEG spectrum, it is possible to identify an individual patient likelihood of experiencing burst suppression at typical anesthetic doses (FIG. 8, right side), versus those with higher anesthetic drug requirements, and a lower likelihood of experiencing burst suppression at typical anesthetic doses (FIG. 8, right side).

Beyond identifying patients with lower drug requirements or a higher likelihood of burst suppression, more formal predictions about a patient's dose response could also be made. In one instance, the alpha power could be used to select, using the methods described above, a pharmacological model that is most appropriate for the patient. This pharmacological model, for instance, could be specified in terms of both pharmacokinetic and pharmacodynamics descriptions, and could be represented mathematically using differential equations describing the time course of drug concentrations and resulting patient states. Similarly, the alpha power could be used to specify or suggest appropriate drug infusion rates, target effect concentrations, or expired anesthetic concentrations, or ranges of such rates, target concentrations, and expired concentrations, appropriate for unconsciousness under general anesthesia, or for sedation.

As described earlier, anesthesia-induced alpha waves have a basic neurophysiological mechanism that is similar to other EEG oscillations, including awake posterior alpha waves and sleep spindles. As a result, other related EEG oscillations recorded pre-operatively could also be used within the context of the describe systems and methods to predict anesthetic drug requirements, likelihood of burst suppression, and to select anesthetic pharmacological models or administration levels or ranges. Moreover, because alpha waves are related to cognition, cognitive testing measurements could also be used within the context of the described system and methods to predict anesthetic drug requirements, likelihood of burst suppression, and to select anesthetic pharmacological models or administration levels or ranges. Moreover, since alpha waves are related broader measures of brain function, any valid measure of brain function might also be conceivably used within the context of the described system and methods to predict anesthetic drug requirements, likelihood of burst suppression, and to select anesthetic pharmacological models or administration levels or ranges. This could include, for instance, prior diagnoses of cognitive impairment, dementia, or neurovascular disease that might influence the brain's response to neuroactive drugs.

Closed-loop control, also referred to as feedback control or automatic control, of anesthesia-induced brain states such as medical coma, general anesthesia, and sedation, rely in part upon dose response models to automatically determine the appropriate dose to achieve a desired patient state. Consequently, the systems and methods described herein could be used to select appropriate dose response models or pharmacological models for use in closed-loop control of anesthesia-induced brain states.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A method for determining a response of a patient to the administration of at least one drug having anesthetic properties, the method comprising:
a) receiving physiological data acquired from a patient;
b) analyzing the physiological data to determine at least one indicator of brain function;
c) determining, based on the at least one indicator of brain function, a response of the patient to the administration of at least one drug having anesthetic properties; and
d) generating a report indicative of the response.

2. The method of claim 1, wherein the method further comprises receiving at least one of electroencephalogram (EEG) data and cognitive testing data.

3. The method of claim 2, wherein the method further comprises analyzing EEG data in step b) to determine the at least one indicator of brain function.

4. The method of claim 3, wherein the method further comprises applying a multitaper technique in the analysis of step b).

5. The method of claim 3, wherein the method further comprises identifying signatures in at least one of an amplitude and a power spectrum corresponding to the EEG data.

6. The method of claim 1, wherein the method further comprises receiving a user input comprising information regarding the patient, the at least one drug, or both.

7. The method of claim 1, wherein the method further comprises generating a pharmacodynamic curve based on the response determined.

8. The method of claim 2, wherein the method further comprises analyzing the physiological data to determine a likelihood of a burst suppression and estimating a drug dose based on the likelihood.

9. The method of claim 1, wherein the method further comprises estimating a drug dose, based on the response determined, for achieving a predetermined state of anesthesia or sedation of the patient.

10. A system for determining a response of a patient to the administration of at least one drug having anesthetic properties, the system comprising:
a plurality of sensors configured to acquire physiological data from the patient;
a processor programmed to execute instructions stored in a non-transitory computer-readable medium to:
i) receive the physiological data;
ii) analyze the physiological data to determine at least one indicator of brain function;
iii) determine, based on the at least one indicator of brain function, a response of the patient to the administration of at least one drug having anesthetic properties;
iv) generate a report indicative of the response; and
an output for providing the report.

11. The system of claim 10, wherein the plurality of sensors is further configured to acquire electroencephalogram (EEG) data from the patient.

12. The system of claim 11, wherein the processor is further programmed to analyze the EEG data to determine the at least one indicator of brain function.

13. The system of claim 12, wherein the processor is further programmed to apply a multitaper technique in the analysis.

14. The system of claim 11, wherein the processor is further programmed to identify signatures in at least one of an amplitude and a power spectrum corresponding to the EEG data.

15. The system of claim 10, wherein the system further comprises a user interface configured to receive an indication of the patient, of the at least one drug, or both.

16. The system of claim 15, wherein the processor is further programmed to determine the response based on the indication.

17. The system of claim 10, wherein the processor is further programmed to generate a pharmacodynamic curve based on the response determined.

18. The system of claim 10, wherein the processor is further programmed to analyze the physiological data to determine a likelihood of a burst suppression, and estimate a drug dose based on the likelihood.

19. The system of claim 10, wherein the processor is further programmed to estimate a drug dose, based on the response determined, to achieve a predetermined state of anesthesia or sedation of the patient.

20. The system of claim 19, wherein the system further comprises a drug delivery system controllable by the processor and configured to deliver to the patient the drug dose estimated.

21. The system of claim 19, wherein the processor is further configured to operate the system in a closed-loop to control the predetermined state of anesthesia or sedation of the patient.
